(19) United States
(12) Patent Application Publication
(10) Pub. No.: US 2023/0407246 A1
(43) Pub. Date: Dec. 21, 2023

(54) METHOD FOR PRODUCING FOOD PRODUCTS USING CELLS GROWN IN CULTURE AS AN ALTERNATIVE TO ANIMAL HUSBANDRY

(71) Applicant: THE REGENTS OF THE UNIVERSITY OF CALIFORNIA, Oakland, CA (US)

(72) Inventor: Brian Maiorella, Oakland, CA (US)

(73) Assignee: THE REGENTS OF THE UNIVERSITY OF CALIFORNIA, Oakland, CA (US)

(21) Appl. No.: 18/354,719

(22) Filed: Jul. 19, 2023

Related U.S. Application Data
(63) Continuation of application No. PCT/US2022/013843, filed on Jan. 26, 2022.
(60) Provisional application No. 63/141,942, filed on Jan. 26, 2021.

(51) Int. Cl. C12N 5/00; A23L 13/00

(52) U.S. Cl. CPC C12N 5/0062; A23L 13/00

(57) ABSTRACT

Systems and methods are provided for producing a product of cells organized into a desired macroscopic three-dimensional structure from one or more cell types with engineered donor and receptor pairs and optional scaffolds. The methods are suited to the production of food products. Because the final product is constructed with the same types of cells that are generally arranged in approximately the same three-dimensional structure as in animal muscle tissue, the final product can replicate the sensory and nutritional profile found in conventional, naturally produced meat.

### CROSS-REFERENCE TO RELATED APPLICATIONS

This application claims priority to, and is a 35 U.S.C. § 111(a) continuation of, PCT international application number PCT/US2022/013843 filed on Jan. 26, 2022, incorporated herein by reference in its entirety, which claims priority to, and the benefit of, U.S. provisional patent application Ser. No. 63/141,942 filed on Jan. 26, 2021, incorporated herein by reference in its entirety. Priority is claimed to each of the foregoing applications.

The above-referenced PCT international application was published as PCT International Publication No. WO 2022/164858 A1 on Aug. 4, 2022, which publication is incorporated herein by reference in its entirety.

### STATEMENT REGARDING FEDERALLY SPONSORED RESEARCH OR DEVELOPMENT Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to methods for assembling macroscopic structures of cells and cell growth supporting culturing systems and methods and more particularly to processes for the production of assembled products such as “cultured-meat” or “clean-meat” using a variety of different cell types grown in culture. More particularly cells of various types are first grown by culture in free suspension in bioreactors, and then assembled into a desired macroscopic structure as driven by multiple docking donor/receptor pairs. Scaffolds and support fibers with open donor or receptors may also be used to orient and organize cells

### 2. Background

There is a very large market demand for meat throughout the world, with the average American individual consuming over 200 pounds of meat per year. Current retail sales of red meat and poultry are approximately $92 billion per year. Since at least as early as 1932, researchers have proposed supplementing or replacing animal husbandry with cell culture for the production of meat for human consumption to meet rising demand. However, despite serious efforts in academia and industry, no such “clean-meat” product has reached the market. Meaningful development of an economical clean meat industry remains extremely challenging.

Conventional approaches for producing a meat substitute using cells grown in culture either use a suspension culture to produce a homogeneous product, which does not meet consumer expectations, or uses organ culture methods that create a product with some desired attributes but at unacceptably high price.

One proposed strategy, for example, was to grow animal cells in free suspension in large bioreactors (i.e., taking advantage of economies of scale of such reactors to control cost), and then to assemble the cells into a macroscopic solid by the application of transglutaminase to catalyze protein crosslinking between cells. However, by indiscriminately assembling all cells, this method resulted in a homogeneous product that lacked the complex macroscopic structure which consumers expect of meat.

In an attempt to overcome the limitations of this method to achieve a desired macroscopic structure, some researchers have pursued tissue engineering techniques to produce an edible “clean-meat.” This involved applying methods pioneered for the growth of tissues or organs for use in research or in human transplantation. In those tissue culture approaches, cells of multiple types (i.e., muscle, fat, connective tissue, etc.) are grown in co-culture and allowed to self-assemble into macroscopic structures during growth in keeping with their natural functions in the tissue (sometimes requiring the presence of a scaffold for assembly and the use of specific growth and differentiation factors to drive the desired assembly behavior). The result was a product that had some of the desired macroscopic structure (i.e., resembling muscle tissue as expected in meat), but the process was subject to very high costs for growth of the cells.

In tissue culture, the cells grow in a packed configuration which limits the diffusion of oxygen and other nutrients. This diffusion limitation imposes a limitation on the size of the culture system (i.e., typically only thin layers or strands of cells can be grown). Complex reactor types such as hollow fiber bioreactors can be used to provide oxygen and nutrients by rapidly circulating culture medium through many embedded semi-porous fibers to allow growth of larger tissues, but the cost of such reactors is high.

Unlike tissue culture methods, growth of cells in suspension can be readily scaled to large volumes in simple tanks as already employed in the biopharmaceutical industry, resulting in increasing economies of scale as bioreactor volume is increased to meet increasing demand. Improvements in bioreactor plant design and cell culture methods for growth of cells in suspension, as well as advances in cellular and tissue engineering techniques will reduce the cost and increase the potential availability of cultured cells. With such advances, there is a need for the development of techniques for the controlled assembly of macrostructures of a variety of cells and structures that replicate the sensory and nutritional profile of conventionally produced meat or similar natural product. Further, such techniques will allow the creation of novel structures of assembled cells.

### BRIEF SUMMARY

Systems and methods are provided for the creation of structured cell products comprising three-dimensional assemblies of one or more types of cells produced by growth in suspension cell culture. For application in the production of a cultured clean meat product, the goal of the methods is to assemble cells in a structure that mimics the spatial structure, cell types, fats and connective tissues of animal muscle cells to produce a cultured meat product. Because the final product is constructed with the same types of cells that are generally arranged in approximately the same three-dimensional structure as animal muscle tissue, the final product can replicate the sensory and nutritional profile found in conventional, naturally produced meat. Further, such techniques will allow the creation of novel structures of assembled cells allowing the exploration of novel food or other cellular products.

In one embodiment, different cells are grown in free suspension, making possible the economies of scale that come in using very large, stirred tank bioreactors. After growth, the cells are readily assembled into the desired macroscopic structure, including features such as fat marbling and muscle fiber orientation as expected by consumers of meat, by simply controlling the conditions under which the desired multiple cell types or cell types and scaffolds are mixed together and dewatered.

In this illustration, cells of various types are first grown by culture in free suspension in bioreactors, and then assembled into a desired macroscopic structure as driven by the binding of docking donor/receptor pairs. Assembly of two cells A and B together, for example, can be achieved by including a display of docking-donor molecules on the surface of cell A and complementary docking-receptor molecules on the surface of cell B. Distinctly different donor/receptor pairs can cause cells of type A and B to preferentially assemble and cells of type C and D to preferentially assemble simultaneously or sequentially. Similarly, cell type E can preferentially assemble with other type E cells by including both donor and matching receptor on the same cell type.

By including multiple docking molecule types on a cell, and regulating the relative surface concentration of the display of receptor types and/or their relative affinities, then cell type A can be made to most preferentially bind to cell type B, while also binding but at lower preference to cell type C.

When the various cell types are mixed in suspension and allowed to flocculate, this directed assembly will result in the formation of macroscopic structure. The arrangement of the various cell types can be controlled based upon the choice and density of docking molecules expressed on each cell type, and on the conditions such as cell concentration, mixing regime, relative binding affinities of the different docking pairs, etc., under which the cells are brought together.

In another embodiment, the docking molecules can be designed so that the docking event is further dependent upon a controllable environmental condition such as being activated by pH, temperature, osmolarity, the addition of a soluble linking adapter [e.g., multivalent receptor molecule which will simultaneously bind to and link the docking donors of two or more cells], or the addition of a chemical linking reagent, etc. The conditional control over docking allows cells of multiple types in the liquid suspension to be readily mixed together before docking is activated, and so that docking of various subsets of cell types can be temporally sequenced to give increased control over the final macroscopic structural arrangement.

In another embodiment, a display of docking molecule receptors and/or donors can be incorporated onto the surface of a scaffold to direct assembly of cells (or a subset of cell types) onto the scaffold, again affording further control over the final macroscopic structure. Likewise, docking receptors can be incorporated onto the cell surface which are complementary to naturally occurring receptors on the scaffold, with the same result of organizing cell assembly to the scaffold.

Docking donor/receptor pairs can be selected from among the many types known in nature (such as a cell surface antigen and complementary cell-surface antibody, cell-surface oligosaccharide and complementary lectin, cell surface receptor and complementary binding partner, zinc finger and complementary nucleic acid, etc.), or can be a protein binding pair specifically engineered for the purpose, or can be any novel molecule pair which can be engineered to be displayed on the cell or scaffold surface and which has the desired selective binding properties.

In one embodiment, genetic engineering techniques can be used to achieve the desired expression of either donor or receptor on one cell type, while relying on naturally occurring matching complement on the binding partner cell or scaffold; or genetic engineering techniques can be used to achieve expression of both donor and receptor. In another embodiment, enzymatic or other chemical reaction mechanism can be used to place binding donor and/or receptor onto the cell or scaffold surface.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a functional block diagram of a method for producing a product of a desired macrostructure using cells grown in culture according to one embodiment of the technology.

FIG. 2 is a schematic diagram depicting a scaffold and two cell types with engineered docking donor-receptor pairs on their surface that drive self-assembly that can be organized into a desired structure according to the technology.

FIG. 3 is a schematic diagram depicting a scaffold and two cell types where the binding of one of the cell types is conditional upon an environmental control factor according to another embodiment of the technology.

FIG. 4 is a diagram of a scaffold and two cell types wherein the binding of one of the cell types is conditional upon addition of a linking adapter according to another embodiment of the technology.

### DETAILED DESCRIPTION

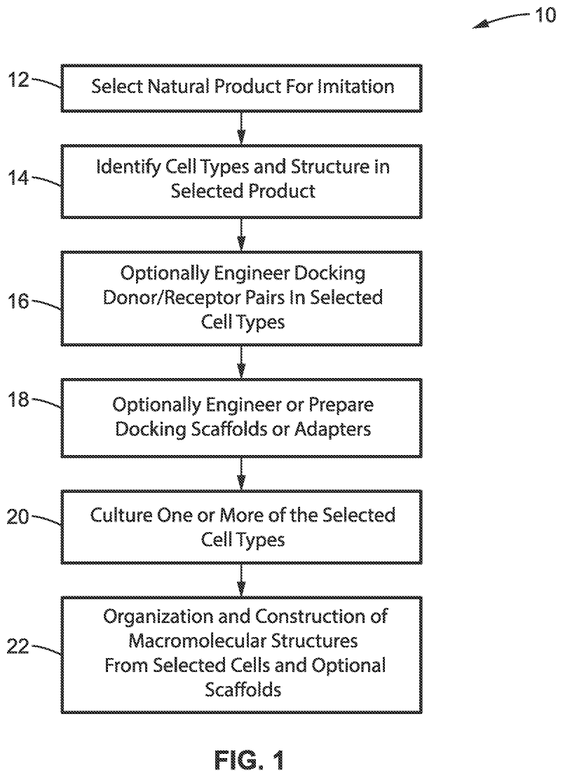

Referring more specifically to the drawings, for illustrative purposes, systems and methods for organizing and constructing macromolecular structures from selected cells and optional scaffolds are generally shown. Several embodiments of the technology are described generally in FIG. 1 through FIG. 4 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

The methods can be adapted to any situation where organization of one or more types of cells into a controlled 3-dimensional structure is desired. However, a “cultured meat” product is used to generally illustrate the methods and resulting macromolecular structure characteristics. In this illustration, animal derived muscle cells, fat cells, and optional scaffolding strands are used to organize muscle like fiber structures that may be assembled into a cultured meat product.

Turning now to FIG. 1, an embodiment of the method 10 for assembling macromolecular structures from cultured cells and scaffolds is shown schematically. At block 12, the product or part of the product that is to be imitated with the methods is selected and the structure analyzed. Natural animal products that have cells that can be cultured are suitable for selection. For example, cells may be selected at block 12 from among those derived from any mammal (including cow, pig, goat, sheep), any poultry (including chicken, duck), any fish (including salmon, tuna, tilapia, pollock, cod, catfish), and any crustacean (including lobster, crab, shrimp).

Cell types, microstructures, macrostructures and overall textures of the selected product are identified and selected for culturing at block 14 of FIG. 1. The identification of the cells and structures of the final product to be imitated at block 14 will provide a framework for the selection of cells and structures to be cultured and assembled and the nature and sequence of the assembly. The specific cell types to be chosen for culturing at block 14 need not be of the identical cell type that was identified as present in the product to be imitated but are cell types selected to imitate the desired features.

The cells to be cultured that are identified and selected at block 14 may come from different sources and the technology can be applied to any cell types which can be grown in suspension culture. Generally, animal cells may be in a pluripotent state, a multipotent state or a specialized or differentiated state during development. Pluripotent cells may differentiate into one of several different cell types in the body; multipotent cells have fewer options for differentiation and specialized cells will only produce one type of cell. For example, the methods could be applied to a primary cell explant from an animal (i.e., a differentiated cell type subject to senescence) which is then adapted to suspension growth (by means of serial culture, or by means of genetic engineering), and which still remains subject to senescence. Likewise, the methods could be applied to continuous (immortal) cell lines of infinite proliferative capacity, and which are adapted to suspension growth.

Any cells that can be propagated in suspension culture can be selected. For animal-derived cells, this can include those primary cells derived directly from an animal and of a type that readily adapt to suspension culture such as lymphocytes, or those stem cells which readily adapt to suspension culture. They can also be cells derived from the differentiation of stem cells and that retain the ability to readily adapt to suspension culture. Alternatively, the selected animal cells may be cells that have been converted to a suspension culture phenotype through genetic engineering, or animal cells which have been converted to a suspension culture phenotype through conventional means of transformation (such as radiation, or chemical mutagenesis, or viral transformation).

The cell types used to produce a final product need not be limited to a single species. As an example, a hybrid product could incorporate both animal cells and yeast (or other fungal) cells to create a hybrid food product of desired structure and nutritive features.

The cells identified and selected for culturing at block 14 may optionally be engineered to possess and display one or more types of surface donor/receptor pairs at block 16.

There are many different types of docking donor/receptor pairs available for engineering at block 16 that will provide controllable cell-to-cell or cell-to-scaffold coupling. The selection of one or more types of docking pairs may be a novel molecule pair which has been engineered to be displayed on the cell surface and that has the desired selective binding properties. Docking donor/receptor pairs may also be selected from among the many types of docking pairs known in nature such as a cell surface antigen and complementary cell-surface antibody, a cell-surface oligosaccharide and complementary lectin, a cell surface receptor and complementary binding partner, a zinc finger and a complementary nucleic acid, etc., or can be a protein donor/receptor binding pair specifically engineered for the purpose. Docking donor/receptor pairs may also be selected from complementary binding partners where the donor or receptor is a naturally occurring feature of the cell or scaffold such as a cell-surface antibody and a complementary natural scaffold-feature antigen or vice versa. For example, if a cell is engineered to have a surface antibody which will bind to a natural surface antigen on a scaffold, then this could allow for the formation of a desired fiber structure.

Known genetic engineering techniques can be used to achieve expression of both donor and receptor structures on the cell surfaces. For example, in various embodiments, at least one of the binding donors or receptors may be introduced to cells by genetic engineering of the cell to express the donor or receptor, or by genetic engineering of the cell to upregulate expression and/or cell surface display of the engineered donor or receptor.

In another embodiment, enzymatic or other chemical reaction mechanisms can be used to place binding donor and/or receptor structures onto the cell surface of selected cells or scaffolds. Genetic engineering techniques can also be used at block 16 to achieve the desired expression of either donor or receptor on one cell type, while relying on naturally occurring matching complement on the binding partner cell or scaffold.

By including multiple docking molecule types on a cell or scaffold and regulating the relative surface concentration of the docking donor or receptor types and/or their relative affinities, then a first cell type can be made to preferentially bind to a second cell type or scaffold type, while also binding but at lower preference to a third cell type or scaffold and so forth.

In another embodiment, the docking molecules can be designed so that the docking event is further dependent on a controllable environmental condition such as being activated by pH, temperature, or osmolarity. As an example, the docking donor/receptor pair may be chosen such that a shift in pH alters the conformation of the donor or receptor and wherein binding is favored at the conformation of one pH and is less favored at the conformation of a different pH.

A secondary environmental condition control may also include the addition of a soluble linking adapter e.g., multivalent receptor molecule which will simultaneously bind to and link the docking donors of two or more cells. Another secondary environmental condition control may also include the addition of a chemical linking reagent, etc.

The engineered primary and secondary controls allow the temporal control over the activation of the docking events of various subsets of cell types and the sequence of docking events to give increased control over the final macroscopic structural arrangement of the final product.

In addition to engineering docking pairs in cells at block 16, one or more types of cell scaffolds can be optionally selected and engineered at block 18. The selected scaffolds at block 18 can be engineered to have docking molecule receptors and/or donors incorporated onto the surface of a scaffold to direct assembly of cells (or a subset of cell types) onto the scaffold, affording further control over the final macroscopic structure of the product. Likewise, docking donors or receptors can be incorporated onto the cell surface which are complementary to naturally occurring binding partners on the scaffold at block 18, with the same result of organizing cell assembly to the scaffold.

A long linear chain scaffold can be used, for example, to arrange cells of the chosen type at block 16 that express a complementary binding partner to that engineered on the scaffold into a long linear fiber strand of cells. Additional cells can then be organized by directed binding around this “seed” fiber. Conventional methods of the food processing industry can be used to further orient collections of linked cells relative to each other such as by application of shear during a mixing or dewatering process.

Some examples of possible scaffold and linking adapter backbones for use at block 18 include, but are not limited to: gelatin, alginate, agarose, chitosan, amylose, amylopectin, glycogen, dextran, cellulose and other polysaccharides; fibrin, collagen, elastin, laminin, and other proteins, glycoproteins and proteoglycans and derivatives thereof. It will be understood, however, that the scaffold material might not be a highly refined chemical, rather it may be a natural material such as a plant material from agriculture that includes these chemical types. So, a raw plant fiber such as one including cellulose or starch, or a partially refined plant material such as textured soy or pea proteins may serve this role.

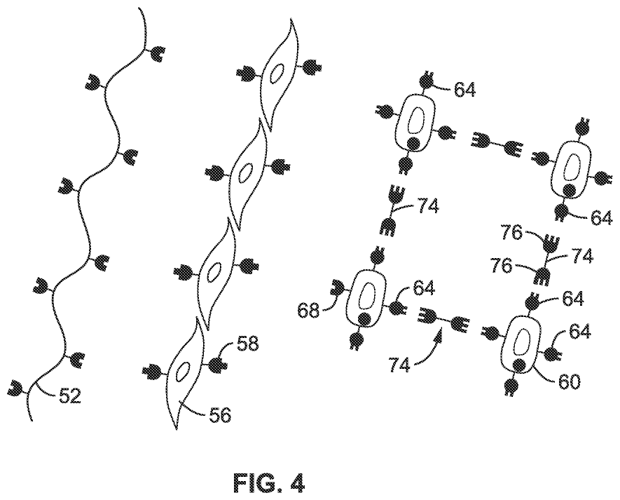

In another embodiment, a linking adaptor molecule may also be identified and optionally engineered at block 18. The linking adaptor structures preferably include at least two donors or two receptors that are complementary binding partners to the natural or engineered donors or receptors of cells that function to link single cell types together as illustrated in FIG. 4. In another embodiment, the linking adaptor structures preferably include at least two donors or two receptors that are complementary binding partners to the donor or receptors of a scaffold and a cell. In another embodiment, the linking adaptor structures preferably include at least one donor and one receptor that are complementary binding partners to receptors or donors of two different cells or scaffolds and cells. These adaptor structures allow the controlled coupling of two different cell types.

Once the selected cells are acquired at block 14 and optionally engineered and acquired at block 16, and, after the optional scaffolds or adaptor links are acquired and engineered at block 18, the different types of cells are then preferably cultured in suspension culture bioreactors at block The cell type or engineered cell types that are selected will guide the culture media composition and culture process conditions that are utilized at block 20. With large scale production schemes, cellular proliferation of selected cells will typically take many generations of exponential growth in a bioreactor to produce pools of individual cell types that are available for organization. The pools of cultured cells that are used for components for organization at block 22 may be undifferentiated or differentiated cells. The cellular proliferation processes at block 20 may also need to be followed by a process of differentiation before organization at block 22 or after. This differentiation process is applied at block 20 or block 22 if it is required to induce the cultured cells to differentiate into the final cell types that make up the final product such as fat cells, muscle cells, fibroblast/connective tissue cells, etc.

The pools of cultured cells, engineered cells and scaffolds that have been prepared are then assembled into macroscale structures at block 22. In one embodiment, the macroscale structures may be assembled into larger groupings or composite structures at block 22.

It can be seen that many different combinations of components and features can be planned and assembled with the methods. Generally, various cell types are mixed and allowed to flocculate. Directed assembly will result in the formation of macroscopic structure where the arrangement of the various cell types can be controlled based upon 1) the sequence of addition of the various cells, scaffolds, docking adapters; 2) the choice and density of docking molecules expressed on each cell type and scaffold type; and 3) on the conditions such as cell concentration, mixing regime, relative binding affinities of the different docking pairs, etc. of assembly when the cells are brought together.

One simple assembly at block 22 is achieved by mixing cells of a single cell type that displays both docking donor and receptor structures on the surface of the cells allowing the cells to couple into a mass. Another simple assembly at block 22 is the docking of two different cell types.

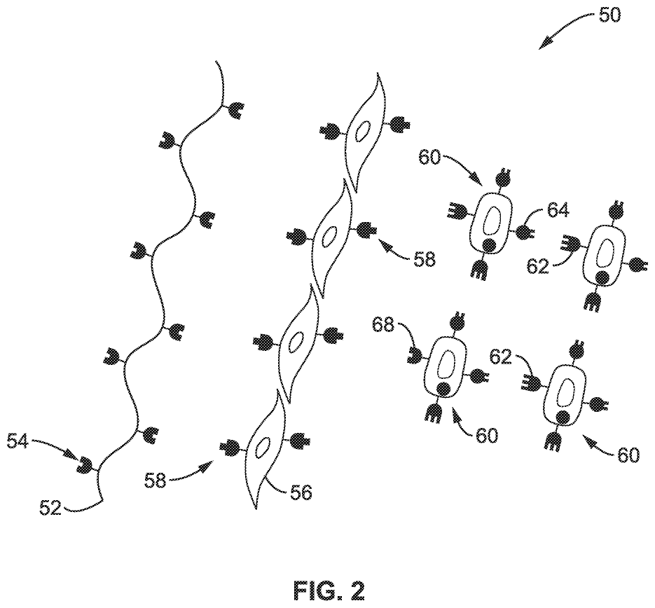

Referring also to FIG. 2, the assembly at block 22 can include scaffolds that assist in the formation of three-dimensional structures of cells. Scaffolding support structures can also assist in the process of differentiation of some cells. As illustrated in FIG. 2, engineered docking donor/receptor pairs and scaffold will drive the self-assembly or the sequential-assembly of a final three-dimensional product. Donor/receptor pairs generally refer to complimentary binding partners that are present on cells or scaffolds and the location of the donor or the receptor on features is normally interchangeable between cells or scaffolds.

The assembly scheme 50 begins with a scaffold strand 52 such as a linear collagen strand, which has been engineered to have donors or receptors. Alternatively, the donors or receptors may be naturally occurring donors or receptors that are typically part of the scaffold 52. In the illustration shown in FIG. 2, the donor 54 of the docking structure is paired with a receptor 58 on the surface of muscle cells 56. It can be seen that the scaffold strand 52 organizes and orients the muscle cells 56 into a linear fiber of cells with the binding of the donors 54 and receptors 58.

Also shown in FIG. 2 is a second set of cells 60, such as fat cells, which have two types of docking pairs on the cell surface. The first docking pair 62, 64 are present in a higher frequency than the second docking donor 68 of the cells 60. In this configuration, the docking pairs that are present in a higher frequency will couple one cell 60 to one or more second cell 60 of the same type. This feature organizes the second set of cells together. However, some cells 60 also have a donor 68 that is the binding partner of the docking receptor 58 of the first set of cells 56. This configuration allows control of the location of the second set of cells 60 with respect to the first set of cells 56 as well as the organization of the second set of cells. In other words, the docking pair configuration allows the location of fat marbling along the organized muscle cells 56 and scaffold 52. In this illustration, simply mixing together and dewatering the culture broths of multiple cell types and scaffold can organize the desired meat structure.

Further in the illustration of FIG. 2, it will be recognized that if the cells of type 60 are expressing the docking pairs 62 and 64 while the cells are being grown in suspension culture, then these cells of type 60 will have a propensity to bind together into clumps during their growth in the bioreactor. If it is desired to avoid cell necrosis at the center of such clumps during growth, then the size of the clumps should be limited during growth to avoid nutrient limitation at the clump centers. Typically, such clumps should be limited to a clump diameter of less than 10 cell diameters, and preferably less than 6 cell diameters during growth. This clumping diameter can be controlled by limiting the binding affinity of the docking pair 62 and 64; and by adjusting the conditions of agitation in the bioreactor of the growing cell culture. Increasing agitation reduces average clump diameter.

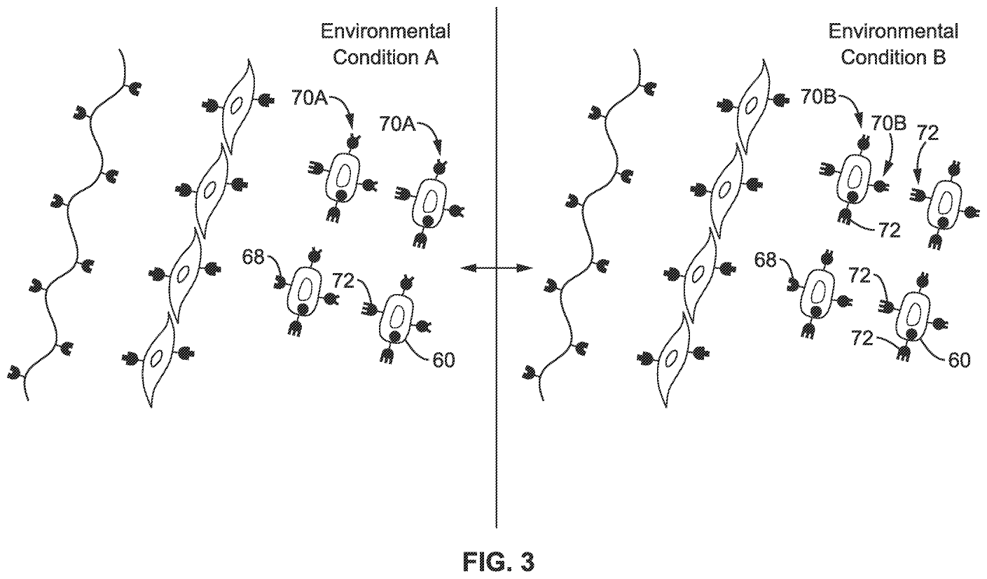

In another embodiment, the coupling of certain docking donor/receptor pairs on one or more cell types is temporally controlled to allow the binding of cells to take place in a sequence. As shown in FIG. 3, the binding affinity of a docking donor/receptor pair can be made conditional upon an environmental condition such as pH, temperature or osmolarity. In this illustration, a shift in the environmental condition controls a shift in conformation of one of the binding partners. Here, the donor of the cell 60 is in conformation 70A under environmental condition A (left side of FIG. 3) and shifts to conformation 70B under environmental condition B (right side of FIG. 3). In environmental condition A, the conformation 70A of the donor is one that does not allow binding to the corresponding partner receptor 72 and so no cell-to-cell binding takes place as a result. However, cell-to-cell docking can take place with a change in environmental conditions so that the conformation 70B of the donor allows the binding of the donor with the receptor 72 so that binding between the cells 60 will occur.

In this illustration, the cells of type 60 can be grown in a bioreactor at an environmental condition A, which is suitable to cell growth and which does not favor binding of the docking pair 70A and 72. Later when it is desired to assemble the cells, the environmental conditions can be modified to convert the conformation 70A of the donors to confirmation 70B, thereby favoring cell binding via the binding pair 70B and 72. The change of conformation with a change in conditions provides enhanced temporal control over the binding events.

It can be seen that this mechanism can be adapted to settings other than what is shown generally in FIG. 3. For example, the controlled conformation change of a donor or receptor can occur between binding partners located on two different cell types or located on a scaffold and a cell type so that the cell type to cell type or cell type to scaffold can be controlled with a change in environmental conditions.

Another general temporal control for the assembly of cells or cells and scaffolds is through the use of an adaptor link 74 that contains paired donors or receptors or one of each. As shown in FIG. 4, cells of cell type 60 display binding donor 64 but do not display a corresponding binding receptor partner. Hence cells 60 are not capable or directed to spontaneously bind with each other. Now binding of cells 60 to other cells 60 is conditional upon the addition of multivalent linking adapter 74 which provides two binding receptors 76 to affect the binding with partner donor 64 of the cells. Therefore, in this illustration, the cells 60 with the donor 68 and the donor 64 can bind with the muscle cells 56 that have a receptor 58. However, the cells 60 that only have the second donor 64 will not be able to bind another cell of its same type in the absence of an added linker 74. Accordingly, the assembly of the cells 60 to another cell type or to an existing scaffold-cell structure can be separated in time from the assembly of cells 60 with other cells 60 with the addition of the linking adaptor 74. Although an adaptor link 74 with two receptors 76 is shown in the illustration of FIG. 4, the adaptor link 74 can also be configured to have two donors or a donor and receptor. In addition, the adaptor link can be used to bind with corresponding donors or receptors found in two different cell types or scaffolds.

It will be appreciated that the technology described in this disclosure generally also can be used whenever it is desirable to control the assembly of cells from a liquid suspension into a preferred macroscopic organization. The production of a “clean-meat” product (i.e., an edible product comprised of animal cells that were grown in culture) is only one example of how the technology can be used. The final product may be a novel food product comprising a combination of plant-derived or fungal derived and cell-culture-derived components, wherein at least part of the desired product structure is created via the methods described herein.

From the description herein, it will be appreciated that the present disclosure encompasses multiple implementations which include, but are not limited to, the following:

A method for producing a macroscopic structure of cells, the method comprising: growing cells of desired multiple cell types in culture in free suspension in bioreactors; and assembling the cells into a desired macroscopic structure as driven by docking of donor/receptor pairs.

The method of any preceding or following implementation, further comprising: engineering a display of docking donors or receptors of a donor/receptor pair on cells of a first cell type; and engineering a display of complementary docking donors or receptors on cells of a second cell type, the donors or receptors complementary to the docking donor or receptors on the cells of the first cell type; wherein the cells of the first cell type couple to the cells of the second cell type with the docking donor/receptor pairs.

The method of any preceding or following implementation, further comprising: engineering a display of docking receptors of a donor/receptor pair on cells of a third cell type, the receptors complementary to docking donors of the donor/receptor pairs of the second cell type; wherein the cells of the first cell type couple to the cells of the second cell type with binding of the docking donor/receptor pairs of the first cell type; and wherein the cells of the third cell type couple to the cells of the second cell type with the binding of the docking donor/receptor pairs of the second cell type.

The method of any preceding or following implementation, further comprising: engineering a display of docking donors or receptors of a first donor/receptor pair on cells of a first cell type and on cells of a second cell type; and engineering a display of docking donors or receptors of a second donor/receptor pair on the second cell type; wherein, the cells of the first cell type couple to the cells of the second cell type with binding of the first donor/receptor pairs; and wherein, the cells of the second cell type couple to other cells of the second cell type with binding of the second donor/receptor pairs.

The method of any preceding or following implementation, wherein the docking donor and receptor pairs are selected from the group consisting of a cell-surface antigen and complementary cell-surface antibody, a cell-surface oligosaccharide and complementary lectin, a cell surface receptor and complementary binding partner, a zinc finger and complementary nucleic acid, and any engineered protein binding pair.

The method of any preceding or following implementation, the assembly of cells further comprising adding an adaptor with at least two donor or receptor binding sites configured to bind with complementary donors or receptors of the docking donor/receptor pairs.

The method of any preceding or following implementation, further comprising engineering a display of docking donors or receptors of a donor/receptor pair on cells of a first cell type; and engineering scaffolds with a display of docking donors or receptors of the donor/receptor pair of the first cell type; wherein, the cells of the first cell type couple to the scaffold with binding of the donor/receptor pairs.

The method of any preceding or following implementation, wherein scaffolds are selected from the group of scaffolds consisting of gelatin, alginate, agarose, chitosan, amylose, amylopectin, glycogen, dextran, cellulose and derivatives thereof.

The method of any preceding or following implementation, wherein scaffolds are selected from the group of scaffolds consisting of fibrin, collagen, elastin, laminin, proteins, glycoproteins, proteoglycans and derivatives thereof.

The method of any preceding or following implementation, further comprising: engineering a display of docking donors or receptors of a second donor/receptor pair on the cells of a first cell type and on cells of a second cell type; wherein, the cells of the first cell type couple to the scaffold with binding of the first donor/receptor pairs; and wherein, the cells of the first cell type couple to the cells of the second cell type with binding of the second donor/receptor pairs.

The method of any preceding or following implementation, further comprising mixing cells in conditions where binding of donor/receptor pairs is inhibited; and shifting conditions to induce binding of donor/receptor pairs to assemble the cells into a desired macroscopic structure; wherein timing and sequence of donor/receptor binding can be controlled.

The method of any preceding or following implementation, wherein shifted culture conditions are one or more conditions selected from the group of conditions consisting of pH, temperature, and osmolarity.

A method for producing a macroscopic structure of cells, the method comprising: providing one or more cell types capable of cell culture propagation, each cell type having a display of docking donors or docking receptors; engineering scaffolds with a display of docking donors or docking receptors complementary to the docking donors or docking receptors of the one or more cell types; and assembling the cells and scaffolds into a desired macroscopic structure by binding complementary docking donors and docking receptors.

The method of any preceding or following implementation, wherein the donors or receptors of each of the cell types are engineered by a process selected from the group consisting of: genetic engineering of the cell to express the donor or receptor, genetic engineering of the cell to upregulate expression and/or cell surface display of the donor or receptor, and by chemical or enzymatic modification of a cell surface to display the donor or receptor.

The method of any preceding or following implementation, further comprising: engineering a display of docking donors or receptors of a first donor/receptor pair on cells of a first cell type and on cells of a second cell type; and engineering a display of docking donors or receptors of a second donor/receptor pair on the second cell type; wherein, the cells of the first cell type couple to the cells of the second cell type with binding of the first donor/receptor pairs; and wherein, the cells of the second cell type couple to other cells of the second cell type with binding of the second donor/receptor pairs.

The method of any preceding or following implementation, wherein the docking donor and receptor pairs are selected from the group consisting of a cell-surface antigen and complementary cell-surface antibody, a cell-surface oligosaccharide and complementary lectin, a cell surface receptor and complementary binding partner, a zinc finger and complementary nucleic acid, and any engineered protein binding pair.

The method of any preceding or following implementation, wherein the docking donor of the one or more cell types is an antibody configured to bind to a natural surface antigen of the scaffold; wherein the cells and the scaffold assemble to form a fiber structure.

The method of any preceding or following implementation, further comprising: providing at least one cell type with docking donor or docking receptors where binding is inhibited or permissive in culture conditions selected from the group of conditions consisting of pH, temperature, and osmolarity; and shifting culture conditions to induce binding of docking donor and docking receptor pairs to assemble the cells; wherein timing and sequence of donor/receptor binding can be controlled.

The method of any preceding or following implementation, the assembly of cells further comprising adding an adaptor with at least two donor or receptor binding sites configured to bind with complementary donors or receptors of the docking donor/receptor pairs of the one or more cell types or the scaffold.

The method of any preceding or following implementation, the assembly of cells further comprising adding an adaptor with at least two donor or receptor binding sites configured to bind with complementary donors or receptors of the docking donor/receptor pairs of the scaffold.

A method for producing a food product, the method comprising: growing cells of desired multiple cell types in culture in free suspension in bioreactors; and assembling the cells into a desired macroscopic structure as driven by multiple docking donor/receptor pairs.

The method of any preceding or following implementation, wherein the food product is a “clean-meat” food product.

The method of any preceding or following implementation, wherein the food product has at least one characteristic similar to a food product produced by animal husbandry, the characteristic selected from the group consisting of fat marbling and muscle fiber orientation.

The method of any preceding or following implementation, wherein the characteristic is achieved by controlling the conditions under which the desired multiple cell types or cell types and scaffolds are mixed together and dewatered.

As used herein, term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly, the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

1. A method for producing a macroscopic structure of cells, the method comprising:
first growing cells of desired multiple cell types in culture in free suspension in bioreactors; and
then assembling the cells into a desired macroscopic structure as driven by docking of donor/receptor pairs.

2. The method of claim 1, further comprising:
engineering a display of docking donors or receptors of a donor/receptor pair on cells of a first cell type; and
engineering a display of complementary docking donors or receptors on cells of a second cell type, said donors or receptors complementary to the docking donor or receptors on the cells of the first cell type;
wherein said cells of the first cell type couple to the cells of the second cell type with the docking donor/receptor pairs.

3. The method of claim 2, further comprising:
engineering a display of docking receptors of a donor/receptor pair on cells of a third cell type, said receptors complementary to docking donors of said donor/receptor pairs of said second cell type;
wherein said cells of the first cell type couple to the cells of the second cell type with binding of the docking donor/receptor pairs of the first cell type; and
wherein said cells of the third cell type couple to the cells of the second cell type with the binding of the docking donor/receptor pairs of the second cell type.

4. The method of claim 1, further comprising:
engineering a display of docking donors or receptors of a first donor/receptor pair on cells of a first cell type and on cells of a second cell type; and
engineering a display of docking donors or receptors of a second donor/receptor pair on said second cell type;
wherein, said cells of the first cell type couple to the cells of the second cell type with binding of the first donor/receptor pairs; and
wherein, said cells of the second cell type couple to other cells of the second cell type with binding of the second donor/receptor pairs.

5. The method of claim 1, wherein the docking donor and receptor pairs are selected from the group consisting of: a cell-surface antigen and complementary cell-surface antibody, a cell-surface oligosaccharide and complementary lectin, a cell surface receptor and complementary binding partner, a zinc finger and complementary nucleic acid, and any engineered protein binding pair.

6. The method of claim 1, said assembly of cells further comprising:
adding an adaptor with at least two donor or receptor binding sites configured to bind with complementary donors or receptors of said docking donor/receptor pairs.

7. The method of claim 1, further comprising:
engineering a display of docking donors or receptors of a donor/receptor pair on cells of a first cell type; and
engineering scaffolds with a display of docking donors or receptors of the donor/receptor pair of the first cell type;
wherein, said cells of the first cell type couple to the scaffold with binding of the donor/receptor pairs.

8. The method of claim 7, wherein scaffolds are selected from the group of scaffolds consisting of gelatin, alginate, agarose, chitosan, amylose, amylopectin, glycogen, dextran, cellulose and derivatives thereof.

9. The method of claim 7, wherein scaffolds are selected from the group of scaffolds consisting of fibrin, collagen, elastin, laminin, proteins, glycoproteins, proteoglycans and derivatives thereof.

10. The method of claim 7, further comprising:
engineering a display of docking donors or receptors of a second donor/receptor pair on said cells of a first cell type and on cells of a second cell type;
wherein, said cells of the first cell type couple to the scaffold with binding of the first donor/receptor pairs; and
wherein, said cells of the first cell type couple to the cells of the second cell type with binding of the second donor/receptor pairs.

11. The method of claim 1, further comprising:
mixing cells in conditions where binding of donor/receptor pairs is inhibited; and
shifting conditions to induce binding of donor/receptor pairs to assemble the cells into a desired macroscopic structure;
wherein timing and sequence of donor/receptor binding can be controlled.

12. The method of claim 11, wherein shifted culture conditions are one or more conditions selected from the group of conditions consisting of pH, temperature, and osmolarity.

13. A method for producing a macroscopic structure of cells, the method comprising:
providing one or more cell types capable of cell culture propagation, each cell type having a display of docking donors or docking receptors;
engineering scaffolds with a display of docking donors or docking receptors complementary to the docking donors or docking receptors of the one or more cell types; and
assembling the cells and scaffolds into a desired macroscopic structure by binding complementary docking donors and docking receptors.

14. The method of claim 13, wherein said donors or receptors of each of said cell types are engineered by a process selected from the group consisting of: genetic engineering of the cell to express said donor or receptor, genetic engineering of the cell to upregulate expression and/or cell surface display of said donor or receptor, and by chemical or enzymatic modification of a cell surface to display said donor or receptor.

15. The method of claim 13, further comprising:
engineering a display of docking donors or receptors of a first donor/receptor pair on cells of a first cell type and on cells of a second cell type; and
engineering a display of docking donors or receptors of a second donor/receptor pair on said second cell type;
wherein, said cells of the first cell type couple to the cells of the second cell type with binding of the first donor/receptor pairs; and
wherein, said cells of the second cell type couple to other cells of the second cell type with binding of the second donor/receptor pairs.

16. The method of claim 13, wherein the docking donor and receptor pairs are selected from the group consisting of: a cell-surface antigen and complementary cell-surface antibody, a cell-surface oligosaccharide and complementary lectin, a cell surface receptor and complementary binding partner, a zinc finger and complementary nucleic acid, and any engineered protein binding pair.

17. The method of claim 13, wherein said docking donor of said one or more cell types is an antibody configured to bind to a natural surface antigen of said scaffold;
wherein said cells and said scaffold assemble to form a fiber structure.

18. The method of claim 13, further comprising:
providing at least one cell type with docking donor or docking receptors where binding is inhibited or permissive in culture conditions selected from the group of conditions consisting of pH, temperature, and osmolarity; and
shifting culture conditions to induce binding of docking donor and docking receptor pairs to assemble the cells;
wherein timing and sequence of donor/receptor binding can be controlled.

19. The method of claim 13, said assembly of cells further comprising:
adding an adaptor with at least two donor or receptor binding sites configured to bind with complementary donors or receptors of said docking donor/receptor pairs of said one or more cell types or said scaffold.

20. The method of claim 13, said assembly of cells further comprising:
adding an adaptor with at least two donor or receptor binding sites configured to bind with complementary donors or receptors of said docking donor/receptor pairs of said scaffold.

21. A method for producing a food product, the method comprising: growing cells of desired multiple cell types in culture in free suspension in bioreactors; and assembling the cells into a desired macroscopic structure as driven by multiple docking donor/receptor pairs.

22. The method of claim 21, wherein the food product is a cultured meat food product.

23. The method of claim 21, wherein the food product has at least one characteristic similar to a food product produced by animal husbandry, said characteristic selected from the group consisting of fat marbling and muscle fiber orientation.

24. The method of claim 23, wherein said characteristic is achieved by controlling the conditions under which the desired multiple cell types or cell types and scaffolds are mixed together and dewatered.
